(19) United States
(12) Patent Application Publication
(10) Pub. No.: US 2018/0078418 A1
(43) Pub. Date: Mar. 22, 2018

(54) SYSTEMS AND METHODS FOR LASER PULSE ENERGY CONTROL

(71) Applicant: AMO Development, LLC, Santa Ana, CA (US)

(72) Inventors: Ihor BEREZHNYY, Los Gatos, CA (US); Mark K. LEE, San Jose, CA (US)

(73) Assignee: AMO Development, LLC, Santa Ana, CA (US)

(21) Appl. No.: 15/706,252

(22) Filed: Sep. 15, 2017

Related U.S. Application Data
(60) Provisional application No. 62/395,448, filed on Sep. 16, 2016.

(51) Int. Cl. A61F 9/008

(52) U.S. Cl. CPC A61F 9/00814; A61B 2017/00154; A61F 9/00804

(57) ABSTRACT

A laser pulse energy control system which includes a laser source and a beam divider positioned to receive a calibration laser pulse produced by the laser source. The beam divider reflects a first portion of the calibration laser pulse along a first optical path toward a first plane and transmits a second portion of the calibration laser pulse along a second optical path toward a second plane. An energy meter determines an energy of the first portion of the calibration laser pulse at the first plane and a fluence profiler determines a fluence profile of the second portion of the calibration laser pulse at the second plane. The processor controls an energy of an ablation laser pulse produced by the laser source based on the fluence profile of the second portion of the calibration laser pulse and the energy of the first portion of the calibration laser pulse.

### CROSS REFERENCE TO RELATED APPLICATION

This application claims the benefit of U.S. Provisional Patent Application No. 62/395,448 filed Sep. 16, 2016, which is incorporated by reference as if fully set forth.

### SUMMARY

Laser vision treatment systems are typically used to treat patients by using laser pulses to reshape the cornea of a patient's eye's to improve visual acuity Effective treatment relies, in part, on the energy of the laser pulses be applied to the patient. Incorrect pulse energy can change the planned treatment outcome or make it otherwise difficult to obtain an effectively therapeutic outcome for the patient. Accordingly, delivering a treatment to a patient typically includes setting a desired energy for a laser pulse at a treatment plane.

Conventional methods and systems for setting the pulse energy typically includes a series of manual energy measuremets of a laser pulse and continuously adjusting the energy setting based on the previous energy measurement. These conventional techniques are cumbersome, time consuming and error prone, relying on personnel with specific expertise and training. For example, optical power (e.g., via a lensometer) assessments are conducted by an operator, which are prone to human error. In addition, the conventional techniques include several iterations of adjustment (typically requiring tens of minutes to complete) until a target energy is obtained for achieving a desired optical power. The techniques are also costly, for example, requiring non-reusable plastic cards, expensive equipment, such as lensometers, and the costs to train the operators (typically requiring many hours of training).

Embodiments described herein provide efficient, automated methods and systems for controlling the energy of a laser pulse based on the measured energy and the fluence profile of a calibration laser pulse. These methods and systems may enable setting the correct target laser pulse energy for achieving a desired treatment outcome regardless of the level of fluence non-uniformity

Embodiments described herein provide a laser pulse energy control system which includes a laser source and a beam divider positioned to receive a calibration laser pulse produced by the laser source. The beam divider is configured to reflect a first portion of the calibration laser pulse along a first optical path toward a first plane and transmit a second portion of the calibration laser pulse along a second optical path toward a second plane. An energy meter is configured to determine an energy of the first portion of the calibration laser pulse at the first plane and a fluence profiler configured to determine a fluence profile of the second portion of the calibration laser pulse at the second plane. A processor is configured to control an energy of an ablation laser pulse produced by the laser source based on the fluence profile of the second portion of the calibration laser pulse and the energy of the first portion of the calibration laser pulse.

Embodiments described herein provide a laser pulse energy control method which includes receiving, at a processing device, an energy value corresponding to an energy, determined at a first plane, of a first divided portion of a calibration laser pulse generated by a laser source. The method also includes receiving, at the processing device, fluence profile information corresponding to a fluence profile, determined at a second plane, of a second divided portion of the calibration laser pulse generated by the laser source. The method also includes controlling, by the processing device, an energy of an ablation laser pulse produced by the laser source based on the fluence profile of the second divided portion of the calibration laser pulse and the energy of the first divided portion of the calibration laser pulse.

Embodiments described herein also provide a computer processing device which includes memory configured to store energy values and fluence profile information and a processor. The processor is configured to receive an energy value corresponding to an energy, determined at a first plane, of a first divided portion of a calibration laser pulse generated by a laser source and receive fluence profile information corresponding to a fluence profile, determined at a second plane, of a second divided portion of the calibration laser pulse generated by the laser source. The processor is also configured to control an energy of an ablation laser pulse produced by the laser source by determining a target energy based on the fluence profile of the second portion of the calibration laser pulse and the energy of the first portion of the calibration laser pulse. The processor is further configured to generate a control signal for causing the laser source to produce the ablation laser pulse having the target energy. In response to receiving the control signal, the energy of the ablation laser pulse is controlled by one of: (i) adjusting parameter settings such that the energy of the ablation laser pulse is adjusted to the target energy; and (ii) maintaining the parameter settings such that the energy of the ablation laser pulse is maintained at the target energy.

### BRIEF DESCRIPTION OF THE DRAWINGS

A more detailed understanding can be had from the following description, given by way of example in conjunction with the accompanying drawings wherein:

FIG. 1 illustrates an example laser ablation system for use with embodiments described herein;

FIG. 2 illustrates an example computing environment for use with embodiments described herein;

FIG. 3A illustrates an example wavefront measurement system in which embodiments described herein may be implemented;

FIG. 3B illustrates another example wavefront measurement system in which embodiments described herein may be implemented;

FIG. 4 graphically illustrates cross sections of different exemplary fluence profiles;

FIG. 5 shows an exemplary plastic card with six spherical lenses ablated thereon used with conventional manual measurement systems;

FIG. 6 shows an exemplary lensometer used with conventional manual measurement systems to measure optical power (OP) of a spherical lens ablated on a plastic card;

FIG. 7 illustrates an exemplary sequence of slab diameters that may be used to ablate a spherical lens with an optical power according to an embodiment;

FIG. 8 is a schematic diagram illustrating an exemplary laser vision treatment system in which embodiments described herein may be implemented;

FIG. 9 illustrates an exemplary camera-based fluence profiler and an exemplary energy meter according to embodiments described herein;

FIG. 10 shows an exemplary fluence profile of an individual laser pulse;

FIG. 11 is a flow diagram illustrating an example method of controlling the energy of a laser pulse according to an embodiment;

FIG. 12 is a flow diagram illustrating an example method of determining a target energy of a laser pulse according to embodiments;

FIG. 13A graphically illustrates a cross section of an example fluence profile having a substantially flat shape within the spot area;

FIG. 13B graphically illustrates a computational spherical lens constructed according to the fluence profile shown in FIG. 13A;

FIG. 14A graphically illustrates a cross section of an example fluence profile having a concave shape within the spot area;

FIG. 14B graphically illustrates a computational spherical lens constructed according to the fluence profile shown in FIG. 14A;

FIG. 15A graphically illustrates a cross section of an example fluence profile having a convex shape within the spot area

FIG. 15B graphically illustrates a computational spherical lens constructed according to the fluence profile shown in FIG. 15A;

FIG. 16A graphically illustrates a cross section of an example fluence profile having a substantially flat shape within the spot area;

FIG. 16B graphically illustrates a computational spherical lens obtained after determining a target energy according to an embodiments described herein;

FIG. 17A graphically illustrates a cross section of an example fluence profile having a concave shape within the spot area;

FIG. 17B graphically illustrates a computational spherical lens obtained after determining a target energy according to embodiments described herein;

FIG. 18A graphically illustrates a cross section of an example fluence profile having a convex shape within the spot area;

FIG. 18B graphically illustrates a computational spherical lens obtained after determining a target energy according to an embodiments described herein;

FIG. 19A graphically illustrates a cross section of an example fluence profile having a concave shape within the spot area;

FIG. 19B graphically illustrates a single computational slab obtained using the fluence profile shown in FIG. 19A;

FIG. 20A graphically illustrates a cross section of a fluence profile having a convex shape within the spot area; and

FIG. 20B graphically illustrates a single computational slab obtained using the fluence profile shown in FIG. 20A.

### DETAILED DESCRIPTION OF THE PREFERRED EMBODIMENTS

Embodiments described herein facilitate the accuracy and efficacy of laser eye surgical procedures, such as photorefractive keratectomy (PRK), phototherapeutic keratectomy (PTK), and the like. In some instances, embodiments provide enhanced accuracy of refractive procedures that involve removal or sculpting of the cornea or modification of other opthalmological tissues or structures. Hence, while embodiments described herein can be used with laser eye surgery system and methods for treating a cornea of the eye, embodiments described herein may also be used in alternative ablation procedures and other laser or energy delivery processes.

The techniques disclosed herein can be readily adapted for use with existing laser systems. By providing a more accurate (and hence, for example, less variable) methodology for treating optical errors of an eye, embodiments described herein facilitate sculpting of the cornea or other opthalmological tissues so that treated eyes may consistently and reliably receive the desired optical correction resulting in improved vision.

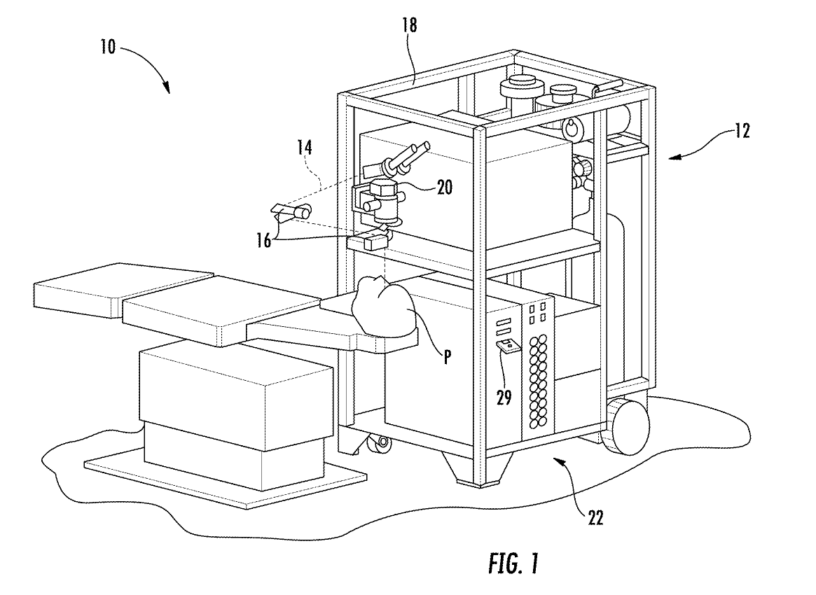

Turning now to the drawings, FIG. 1 illustrates a laser eye surgery system 10. As shown in FIG. 1, the system 10 includes a laser 12 that produces a laser beam 14. Laser 12 is optically coupled to laser delivery optics 16, which directs laser beam 14 to an eye of patient P. A delivery optics support structure (not shown here for clarity) extends from a frame 18 supporting laser 12. A microscope 20 is mounted on the delivery optics support structure, the microscope often being used to image a cornea of the patient's eye.

Laser 12 comprises, for example, an excimer laser, which includes, for example, an argon-fluorine laser producing pulses of laser light having a wavelength of approximately 193 nm. Laser 12 is configured, for example, to provide a feedback stabilized fluence at the patient's eye, delivered via delivery optics 16. Embodiments described herein may be used with alternative sources of ultraviolet or infrared radiation, such as those adapted to controllably ablate the corneal tissue without causing significant damage to adjacent and/or underlying tissues of the eye. Accordingly, alternative to excimer lasers, other types of sources of an ablating beam directed to an eye of a patient include, but are not limited to, solid state lasers and other devices which can generate energy in the ultraviolet wavelength between about 185 and 205 nm and/or those which utilize frequency-multiplying techniques.

Laser system 10 includes a computer system 22. Computer system 22 may include a PC system including user interface devices such as a keyboard, a display monitor, and the like. Computer system 22 may include an input device such as a magnetic or optical disk drive, an internet connection, or the like. Such input devices may be used to download executable programmed instructions from a tangible storage media 29 embodying any of the methods described herein. Tangible storage media 29 may take the form of a floppy disk, an optical disk, a data tape, a volatile or non-volatile memory, RAM, or the like. Computer system 22 may include memory boards and other components for storing and executing programmed instructions. Tangible storage media 29 may optionally embody wavefront sensor data, wavefront gradients, a wavefront elevation map, a treatment map, a corneal elevation map, and/or an ablation table. While tangible storage media 29 will often be used directly in cooperation with an input device of computer system 22, the storage media may also be remotely operatively coupled with processor by means of network connections such as the internet, and by wireless methods such as infrared, Bluetooth, or the like.

Laser 12 and delivery optics 16 will generally direct laser beam 14 to the eye of patient P under the direction of computer system 22. Computer system 22 may selectively adjust laser beam 14 to expose portions of the cornea to the pulses of laser energy so as to effect a predetermined sculpting of the cornea and alter the refractive characteristics of the eye. In some embodiments, both laser beam 14 and the laser delivery optical system 16 are under control of computer system 22 to effect the desired laser sculpting process, with the processor effecting (and optionally modifying) the pattern of laser pulses. The pattern of pulses may by summarized in machine readable data of tangible storage media 29 in the form of a treatment table, and the treatment table may be adjusted according to feedback input into computer system 22 from an automated image analysis system in response to feedback data provided from an ablation monitoring system feedback system. Optionally, the feedback may be manually entered into the computer system by a system operator. Such feedback may be provided by integrating the wavefront measurement system described below with the laser treatment system 10, and computer system 22 may continue and/or terminate a sculpting treatment in response to the feedback, and may optionally also modify the planned sculpting based at least in part on the feedback. Measurement systems are further described in U.S. Pat. No. 6,315,413, the full disclosure of which is incorporated herein by reference.

Laser beam 14 may be adjusted to produce the desired sculpting using a variety of alternative mechanisms. The laser beam 14 may be selectively limited using one or more variable apertures. An exemplary variable aperture system having a variable iris and a variable width slit is described in U.S. Pat. No. 5,713,892, the full disclosure of which is incorporated herein by reference. The laser beam may also be tailored by varying the size and offset of the laser spot from an axis of the eye, as described in U.S. Pat. Nos. 5,683,379, 6,203,539, and 6,331,177, the full disclosures of which are incorporated herein by reference.

Still further alternatives are possible, including scanning of the laser beam over the surface of the eye and controlling the number of pulses and/or dwell time at each location, as described, for example, by U.S. Pat. No. 4,665,913, the full disclosure of which is incorporated herein by reference; using masks in the optical path of laser beam 14 which ablate to vary the profile of the beam incident on the cornea, as described in U.S. Pat. No. 5,807,379, the full disclosure of which is incorporated herein by reference; hybrid profile-scanning systems in which a variable size beam (typically controlled by a variable width slit and/or variable diameter iris diaphragm) is scanned across the cornea; or the like. The computer programs and control methodology for these laser pattern tailoring techniques are well described in the patent literature.

Additional components and subsystems may be included with laser system 10, as should be understood by those of skill in the art. For example, spatial and/or temporal integrators may be included to control the distribution of energy within the laser beam, as described in U.S. Pat. No. 5,646,791, the full disclosure of which is incorporated herein by reference. Ablation effluent evacuators/filters, aspirators, and other ancillary components of the laser surgery system are known in the art. Further details of suitable systems for performing a laser ablation procedure can be found in commonly assigned U.S. Pat. Nos. 4,665,913, 4,669,466, 4,732,148, 4,770,172, 4,773,414, 5,207,668, 5,108,388, 5,219,343, 5,646,791 and 5,163,934, the complete disclosures of which are incorporated herein by reference. Suitable systems also include commercially available refractive laser systems such as those manufactured and/or sold by Alcon, Bausch & Lomb, Nidek, WaveLight, LaserSight, Schwind, Zeiss-Meditec, and the like. Basis data can be further characterized for particular lasers or operating conditions, by taking into account localized environmental variables such as temperature, humidity, airflow, and aspiration.

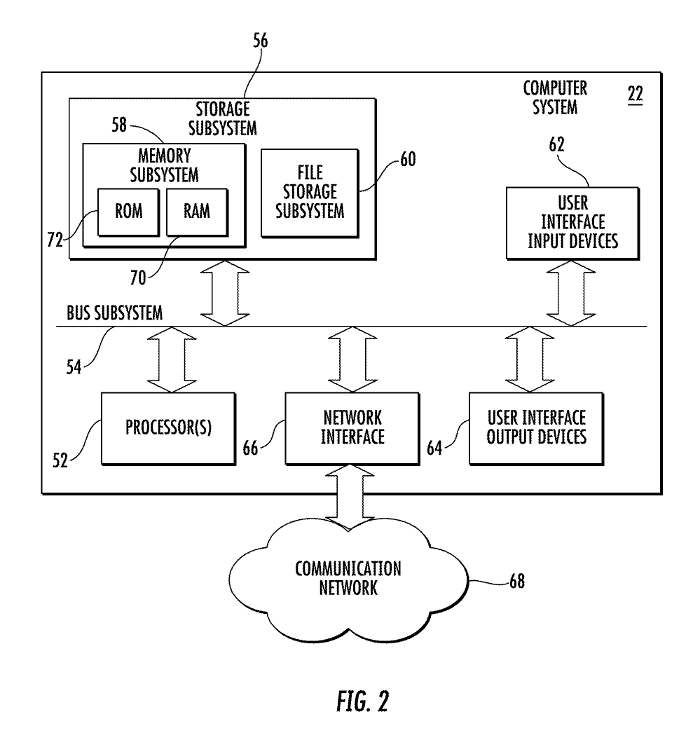

FIG. 2 is a simplified block diagram of exemplary computer system 22 shown in FIG. 1. As shown in FIG. 2, computer system 22 includes at least one processor 52 which may communicate with a number of peripheral devices via a bus subsystem 54. These peripheral devices may include a storage subsystem 56, comprising a memory subsystem 58 and a file storage subsystem 60, user interface input devices 62, user interface output devices 64, and a network interface subsystem 66. Network interface subsystem 66 provides an interface to outside networks 68 and/or other devices, such as the wavefront measurement system 30.

User interface input devices 62 may include a keyboard, pointing devices such as a mouse, trackball, touch pad, or graphics tablet, a scanner, foot pedals, a joystick, a touchscreen incorporated into the display, audio input devices such as voice recognition systems, microphones, and other types of input devices. User input devices 62 will often be used to download a computer executable code from a tangible storage media 29 embodying any of the embodiments described herein. In general, use of the term “input device” is intended to include a variety of conventional and proprietary devices and ways to input information into computer system 22.

User interface output devices 64 may include a display subsystem, a printer, a fax machine, or non-visual displays such as audio output devices. The display subsystem may be a cathode ray tube (CRT), a flat-panel device such as a liquid crystal display (LCD), a projection device, or the like. The display subsystem may also provide a non-visual display such as via audio output devices. In general, use of the term “output device” is intended to include a variety of conventional and proprietary devices and ways to output information from computer system 22 to a user.

Storage subsystem 56 can store the basic programming and data constructs that provide the functionality of the various embodiments described herein. For example, a database and modules implementing the functionality of the embodiments described herein, may be stored in storage subsystem 56. These software modules are generally executed by processor(s) 52. In a distributed environment, the software modules may be stored on a plurality of computer systems and executed by processors of the plurality of computer systems. Storage subsystem 56 typically comprises memory subsystem 58 and file storage subsystem 60.

Memory subsystem 58 typically includes a number of memories including a main random access memory (RAM) 70 for storage of instructions and data during program execution and a read only memory (ROM) 72 in which fixed instructions are stored. File storage subsystem 60 provides persistent (non-volatile) storage for program and data files, and may include tangible storage media 29 (FIG. 1) which may optionally embody wavefront sensor data, wavefront gradients, a wavefront elevation map, a treatment map, and/or an ablation table. File storage subsystem 60 may include a hard disk drive, a floppy disk drive along with associated removable media, a Compact Digital Read Only Memory (CD-ROM) drive, an optical drive, DVD, CD-R, CD-RW, solid-state removable memory, and/or other removable media cartridges or disks. One or more of the drives may be located at remote locations on other connected computers at other sites coupled to computer system 22. The modules implementing the functionality of the embodiments described herein may be stored by file storage subsystem 60.

Bus subsystem 54 provides a mechanism for letting the various components and subsystems of computer system 22 communicate with each other as desired. The various subsystems and components of computer system 22 need not be at the same physical location but may be distributed at various locations within a distributed network. Although bus subsystem 54 is shown schematically as a single bus, alternate embodiments of the bus subsystem may utilize multiple busses.

Computer system 22 itself can be of varying types including a personal computer, a portable computer, a workstation, a computer terminal, a network computer, a control system in a wavefront measurement system or laser surgical system, a mainframe, or any other data processing system. Due to the ever-changing nature of computers and networks, the description of computer system 22 depicted in FIG. 2 is intended only as a specific example for purposes of illustrating one embodiment. Many other configurations of computer system 22 are possible having more or less components than the computer system depicted in FIG. 2.

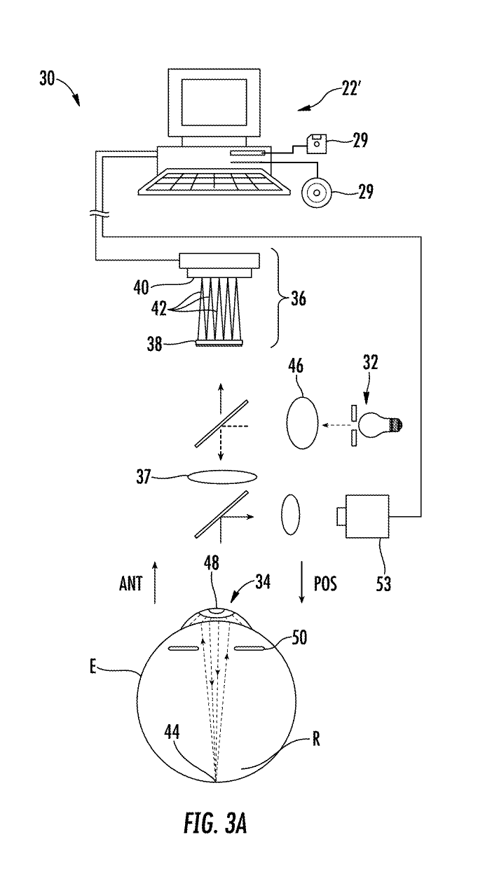

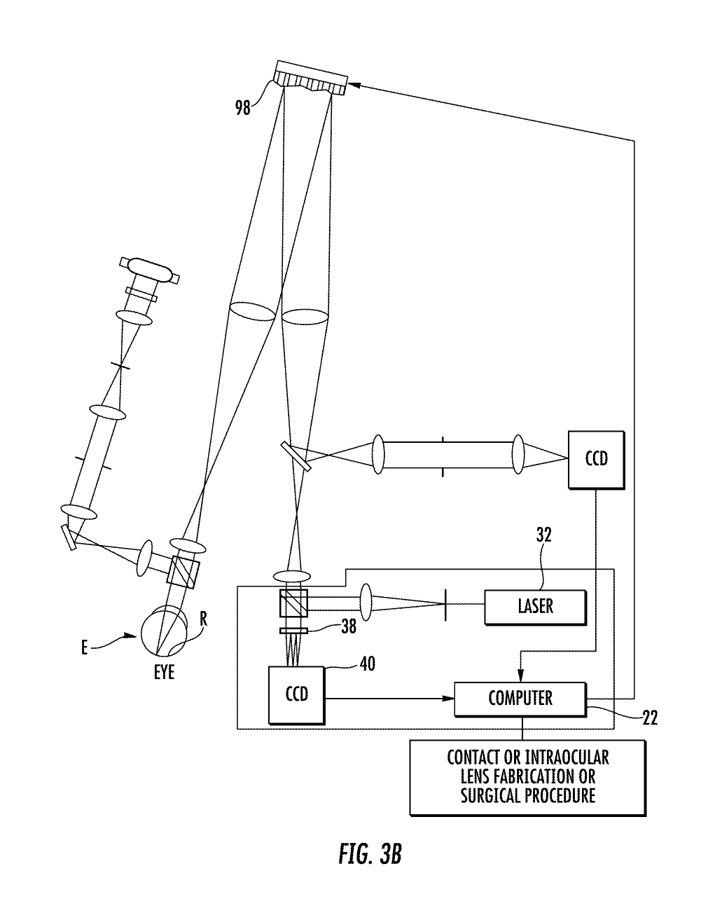

FIG. 3A illustrates an example wavefront measurement system 30 in which embodiments described herein may be implemented. Wavefront measurement system 30 is configured to sense local slopes of a gradient map exiting the patient's eye E. Devices based on the Hartmann-Shack principle generally include a lenslet array to sample the gradient map uniformly over an aperture, which is typically the exit pupil of the eye E. Thereafter, the local slopes of the gradient map are analyzed so as to reconstruct the wavefront surface or map.. In very general terms, wavefront measurement system 30 is configured to sense local slopes of a gradient map exiting the patient's eye E. Devices based on the Hartmann-Shack principle generally include a lenslet array to sample the gradient map uniformly over an aperture, which is typically the exit pupil of the eye E. Thereafter, the local slopes of the gradient map are analyzed so as to reconstruct the wavefront surface or map.

More specifically, one wavefront measurement system 30 includes an image source 32, such as a laser, which projects a source image through optical tissues 34 of eye E so as to form an image 44 upon a surface of retina R. The image from retina R is transmitted by the optical system of the eye (e.g., optical tissues 34) and imaged onto a wavefront sensor 36 by system optics 37. The wavefront sensor 36 communicates signals to a computer system 22′ for measurement of the optical errors in the optical tissues 34 and/or determination of an optical tissue ablation treatment program. Computer 22′ may include the same or similar hardware as the computer system 22 illustrated in FIGS. 1 and 2. Computer system 22′ may be in communication with computer system 22 that directs the laser surgery system 10, or some or all of the components of computer system 22, 22′ of the wavefront measurement system 30 and laser surgery system 10 may be combined or separate. If desired, data from wavefront sensor 36 may be transmitted to a laser computer system 22 via tangible media 29, via an I/O port, via a networking connection 66 such as an intranet or the Internet, or the like.

Wavefront sensor 36 generally comprises a lenslet array 38 and an image sensor 40. As the image from retina R is transmitted through optical tissues 34 and imaged onto a surface of image sensor 40 and an image of the eye pupil P is similarly imaged onto a surface of lenslet array 38, the lenslet array separates the transmitted image into an array of beamlets 42, and (in combination with other optical components of the system) images the separated beamlets on the surface of sensor 40. Sensor 40 typically comprises a charged couple device or “CCD,” and senses the characteristics of these individual beamlets, which can be used to determine the characteristics of an associated region of optical tissues 34. In particular, where image 44 comprises a point or small spot of light, a location of the transmitted spot as imaged by a beamlet can directly indicate a local gradient of the associated region of optical tissue.

Eye E generally defines an anterior orientation ANT and a posterior orientation POS. Image source 32 generally projects an image in a posterior orientation through optical tissues 34 onto retina R as indicated in FIG. 3. Optical tissues 34 again transmit image 44 from the retina anteriorly toward wavefront sensor 36. Image 44 actually formed on retina R may be distorted by any imperfections in the eye's optical system when the image source is originally transmitted by optical tissues 34. Optionally, image source projection optics 46 may be configured or adapted to decrease any distortion of image 44.

In some embodiments, image source optics 46 may decrease lower order optical errors by compensating for spherical and/or cylindrical errors of optical tissues 34. Higher order optical errors of the optical tissues may also be compensated through the use of an adaptive optic element, such as a deformable mirror (described below). Use of an image source 32 selected to define a point or small spot at image 44 upon retina R may facilitate the analysis of the data provided by wavefront sensor 36. Distortion of image 44 may be limited by transmitting a source image through a central region 48 of optical tissues 34 which is smaller than a pupil 50, as the central portion of the pupil may be less prone to optical errors than the peripheral portion. Regardless of the particular image source structure, it will generally be beneficial to have a well-defined and accurately formed image 44 on retina R.

In one embodiment, the wavefront data may be stored in a computer readable medium 29 or a memory of the wavefront sensor system 30 in two separate arrays containing the x and y wavefront gradient values obtained from image spot analysis of the Hartmann-Shack sensor images, plus the x and y pupil center offsets from the nominal center of the Hartmann-Shack lenslet array, as measured by the pupil camera 51 (FIG. 3) image. Such information contains all the available information on the wavefront error of the eye and is sufficient to reconstruct the wavefront or any portion of it. In such embodiments, there is no need to reprocess the Hartmann-Shack image more than once, and the data space required to store the gradient array is not large. For example, to accommodate an image of a pupil with an 8 mm diameter, an array of a 20×20 size (i.e., 400 elements) is often sufficient. As can be appreciated, in other embodiments, the wavefront data may be stored in a memory of the wavefront sensor system in a single array or multiple arrays.

While the methods are generally described with reference to sensing of an image 44, it should be understood that a series of wavefront sensor data readings may be taken. For example, a time series of wavefront data readings may help to provide a more accurate overall determination of the ocular tissue aberrations. As the ocular tissues can vary in shape over a brief period of time, a plurality of temporally separated wavefront sensor measurements can avoid relying on a single snapshot of the optical characteristics as the basis for a refractive correcting procedure. Still further alternatives are also available, including taking wavefront sensor data of the eye with the eye in differing configurations, positions, and/or orientations. For example, a patient will often help maintain alignment of the eye with wavefront measurement system 30 by focusing on a fixation target, as described in U.S. Pat. No. 6,004,313, the full disclosure of which is incorporated herein by reference. By varying a position of the fixation target as described in that reference, optical characteristics of the eye may be determined while the eye accommodates or adapts to image a field of view at a varying distance and/or angles.

The location of the optical axis of the eye may be verified by reference to the data provided from a pupil camera 53. In the exemplary embodiment, a pupil camera 53 images pupil 50 so as to determine a position of the pupil for registration of the wavefront sensor data relative to the optical tissues.

Another example of a wavefront measurement system 31, in which embodiments described herein may be implemented, is illustrated in FIG. 3B. The components of the system 31 in FIG. 3B are similar to those of system 30 in FIG. 3A. System 31 includes an adaptive optical element, however, in the form of a deformable mirror 98. The source image is reflected from deformable mirror 98 during transmission to retina R of eye E The deformable mirror 98 is also along the optical path used to form the transmitted image between retina R and imaging sensor 40. Deformable mirror 98 can be controllably deformed by computer system 22 to limit distortion of the image formed on the retina R or of subsequent images formed of the images formed on the retina R, and may enhance the accuracy of the resultant wavefront data. The structure and use of the system 31 in FIG. 3B are more fully described in U.S. Pat. No. 6,095,651, the full disclosure of which is incorporated herein by reference.

The components of an embodiment of a wavefront measurement system for measuring the eye and ablations may comprise elements of a WaveScan® system, available from AMO Manufacturing USA, LLC, Milpitas, Calif. One embodiment includes a WaveScan system with a deformable mirror as described above. An alternate embodiment of a wavefront measuring system is described in U.S. Pat. No. 6,271,915, the full disclosure of which is incorporated herein by reference. It is appreciated that any wavefront aberrometer could be employed for use with embodiments described herein. Relatedly, embodiments described herein encompass the implementation of any of a variety of optical instruments provided by Abbott Medical Optics, Inc., including the iDesign system, and the like.

Embodiments embodiments described herein may be implemented with any of a variety of optical instruments provided by WaveFront Sciences, Inc., including the COAS wavefront aberrometer, the ClearWave contact lens aberrometer, the CrystalWave IOL aberrometer, and the like. Embodiments described herein may include wavefront measurement schemes such as a Tscherning-based system, which may be provided by WaveFront Sciences, Inc. Embodiments described herein may also include wavefront measurement schemes such as a ray tracing-based system, which may be provided by Tracey Technologies, Corp.

Setting the desired energy for an ultraviolet (UV) laser pulse at the treatment plane is an important step in preparing a laser vision correction system for delivering a treatment to a patient. Having an incorrect pulse energy can change the planned treatment outcome or make it otherwise difficult to obtain an effectively therapeutic outcome for the patient. Setting the energy typically includes energy measurement and energy adjustment based on a last energy measurement. A UV radiation sensor is a device that is used to measure the energy of the UV laser pulse. Embodiments of described herein encompass UV radiation sensors that provide fast (e.g. from milliseconds to microseconds level, depending on laser repetition rate), accurate, and reliable measurements as feedback for energy adjustment controls.

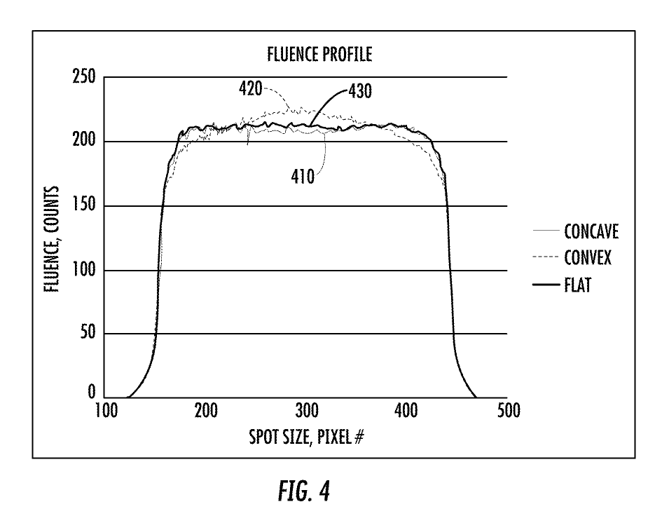

FIG. 4 is a graphical illustration of cross sections of three exemplary fluence profiles 410, 420 and 430. Fluence is defined as the amount of radiation energy delivered per unit area on a surface. Fluence profiles may have different shapes across corresponding laser spots. For example, as shown in FIG. 4, fluence profile 410 has a concave shape across the laser spot, fluence profile 420 has a convex shape across the laser spot and fluence profile 430 has a flat shape across the laser spot. Knowing the fluence profile of a laser pulse is important for many applications. For example, the process of ablation during a LASIK surgery is initiated when the fluence of the laser pulse exceeds a certain threshold. Therefore, depending on the level of fluence non-uniformity, the energy value of the laser pulse may be adjusted to accommodate the fluence non-uniformity to obtain a desired treatment outcome.

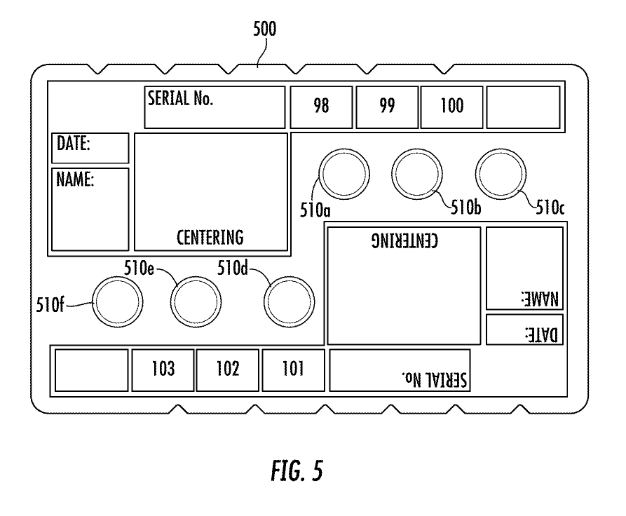

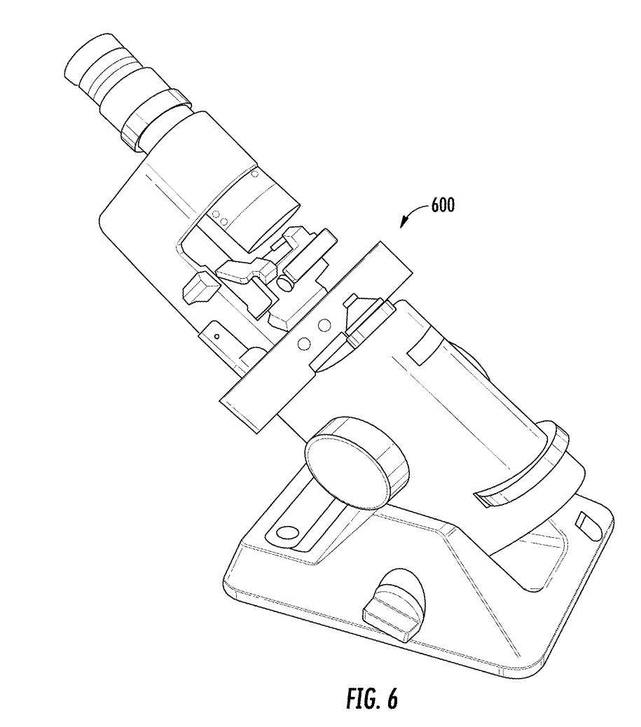

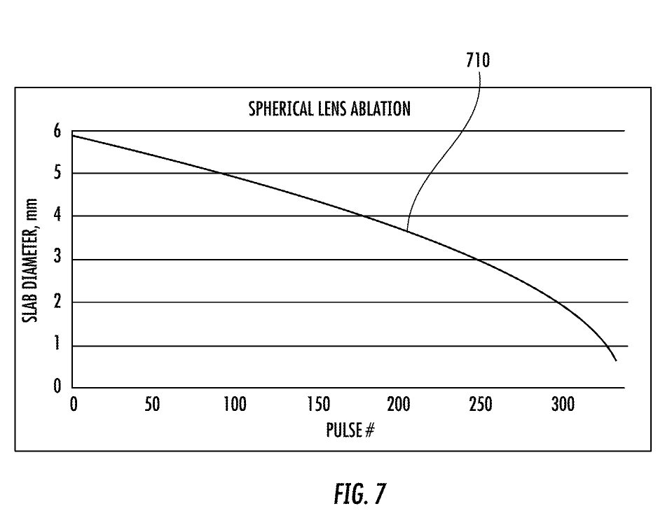

Conventional methods of adjusting the laser pulse energy typically include ablating a spherical lens onto a plastic surface, manually measuring the optical power (OP) of the ablated lens and manually adjusting the pulse energy based on the value of the measured OP using a calibration curve of plastic ablation OP to energy setting. For example, a spherical lens is ablated onto a card. FIG. 5 shows an exemplary plastic card 500 in which six spherical lenses 510a-510f have been ablated onto a surface of the card 500. The number of spherical surfaces shown in FIG. 5 is merely exemplary. Each spherical lens is created by ablating a number of consecutive cylindrical slabs of plastic material of equal thickness and varying diameter. The diameter of each slab is controlled by an iris aperture for the corresponding laser pulse. The OP of an ablated lens is then manually measured for example, using a lensometer, such as lensometer 600 shown in FIG. 6. FIG. 7 graphically illustrates an exemplary slab diameter sequence 710 (measured in mm) used to ablate a spherical lens with a specific optical power (e.g., −4 diopters). The pulse energy is manually adjusted based on the value of the measured OP using a calibration curve of plastic ablation OP to energy setting.

These conventional manual techniques are, however, cumbersome, time consuming and error prone. For example, because the optical power (e.g., via a lensometer) assessments are conducted by an operator, the assessments are error prone to human error. These conventional techniques include several iterations of adjustment (typically requiring tens of minutes to complete) until a target energy is obtained for achieving a desired optical power. In addition, these techniques are often costly (e.g., requiring non-reusable plastic cards, expensive equipment, such as lensometers, and costs to train the operators).

Embodiments described herein include methods and systems for automatically controlling the energy of a laser pulse based on the measured energy and the fluence profile of a calibration laser pulse. These methods and systems facilitate efficiently setting the correct target laser pulse energy for achieving a desired treatment outcome regardless of the level of fluence non-uniformity.

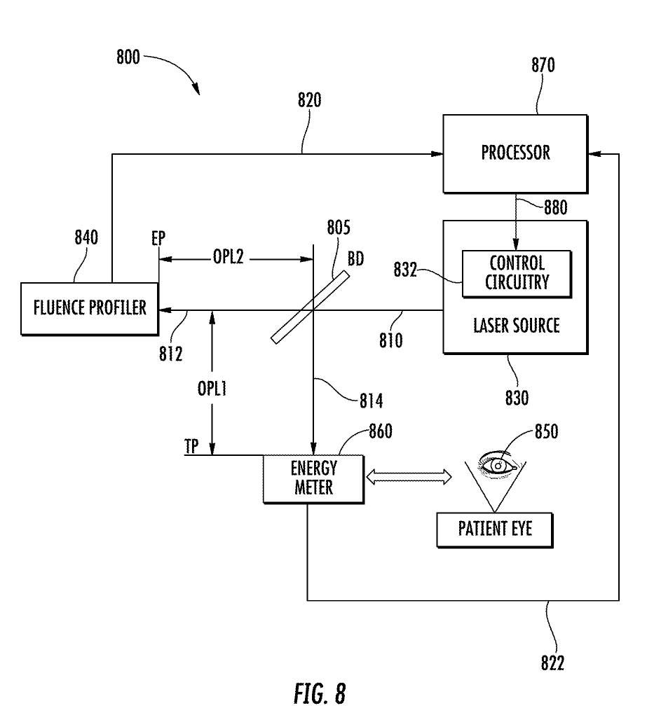

FIG. 8 is a schematic diagram illustrating an exemplary laser vision treatment system 800 in which embodiments described herein may be implemented. As illustrated in FIG. 8, a laser beam 810 is emitted from a laser system 830. The laser system 830 illustrated in FIG. 8 is part of an ophthalmic surgery laser system in which embodiments described herein may be implemented. Embodiments described herein may, however, be implemented in other laser or energy delivery systems where it is desirable to determine beam energy and fluence profile.

As shown in FIG. 8, laser beam 810 is sent through a beam dividing element (i.e., beam divider) 805 such that a first portion 814 of the original laser beam 810 is reflected through a first optical path to be delivered onto the patient's cornea 850 situated at the treatment plane (TP), and a second portion 812 of the beam 810 may be sent to the fluence profiler 840 along a second optical path. In some embodiments, the first and second optical path lengths may be equal. For example, as shown in FIG. 8, the fluence profiler 840 is located at a plane equivalent to the treatment plane (i.e., an equivalent plane), such that a length of the first optical path (OPL1) is equal to the length of the second optical path (OPL2). At the equivalent plane, the laser spot at the fluence profiler 840 may be identical in shape and scaled by a magnitude to the laser spot at the treatment plane (TP). A calibration factor may depend on transmission and reflection properties of the beam dividing element 805. In some embodiments, about 97% of the laser beam 810 propagates toward the treatment plane where the patient's eye is located during a treatment, and about 3% of the laser beam 810 propagates toward the fluence profiler 840. In one embodiment, before the treatment begins, an energy meter 860 is positioned at the treatment plane to measure the energy of a calibration laser pulse.

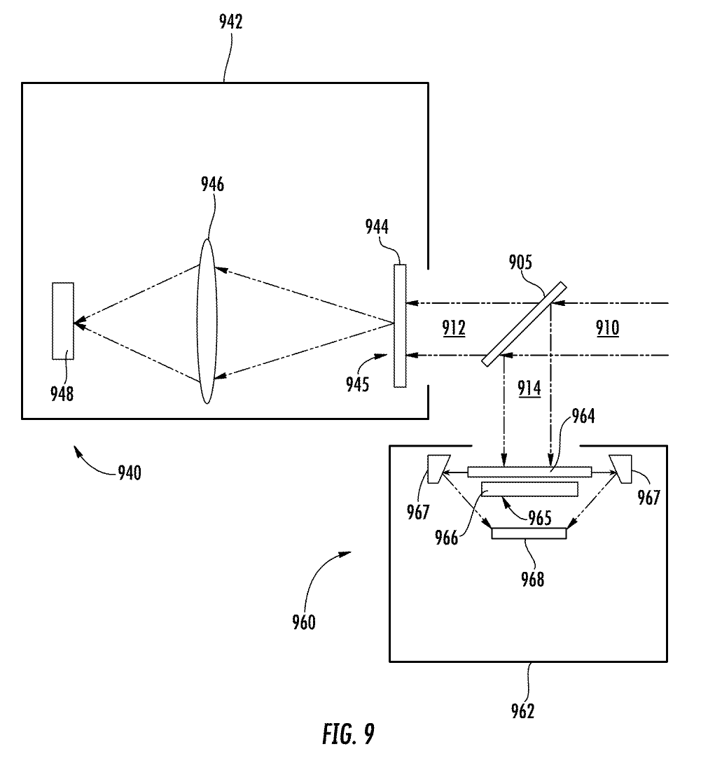

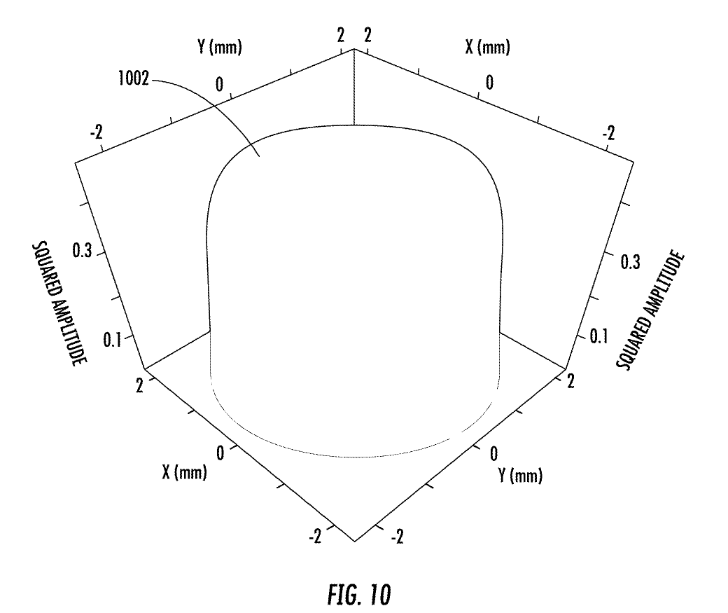

FIG. 9 illustrates an exemplary camera-based fluence profiler 940 and an exemplary energy meter 960 which may be used with embodiments described herein. The fluence profiler 940 may comprise a housing 942 for housing a ultraviolet (UV)-to-visible converter 944, an objective lens 946, and an image sensor 948. The second portion 912 of the laser beam 910 falls on the UV-to-visible converter 944 and excites fluorescent light in the visible range. The florescent light propagates in all directions including directions toward the back surface 945 of the UV-to-visible converter 944. The light emitted from the back surface 945 of the UV-to-visible converter 944 is imaged by the objective lens 946 onto the image sensor 948. The image sensors 948 are configured to detect the profile (shape) of the florescent light that is proportional to the fluence profile of the excitation UV radiation pulse 910. An example three dimensional (3-D) fluence profile of an individual laser pulse 1002 (in mm as a function of the squared amplitude) is shown in FIG. 10.

The energy meter 960 may comprise a housing 962 for housing a UV-to-visible converter 964, a light blocker 966, a conical mirror 967, and a photon detector 968. The first portion 914 of the laser beam 910 falls on the UV-to-visible converter plate 964 and excites fluorescent light in the visible range. The fluorescent light propagates in all directions including directions toward the edge of the UV-to-visible converter 964. The light blocker 966 may be positioned adjacent to a back surface 965 of UV-to-visible converter 964 and may be configured to block ambient light from reaching the photon detector 968. The light blocker 966 may prevent fluorescent light emitted from the back surface 965 of the UV-to-visible converter 964 from traveling past the light blocker 966. The fluorescent light emitted from the edge of the UV-to-visible converter 964 is redirected by the conical mirror 967 toward the photon detector 968, thereby bypassing the light blocker 966. The photon detector 968 may be configured to detect the energy of fluorescent light that is proportional to the total energy of the excitation UV radiation pulse 910.

While exemplary camera-based fluence profiler 940 may be used for detecting the profile of florescent light, other profilers may be used. For example, the second portion 912 of the laser beam 910 may be applied directly to an image sensor (e.g., a charge coupled device (CCD), or the like), or to a pyroelectric camera.

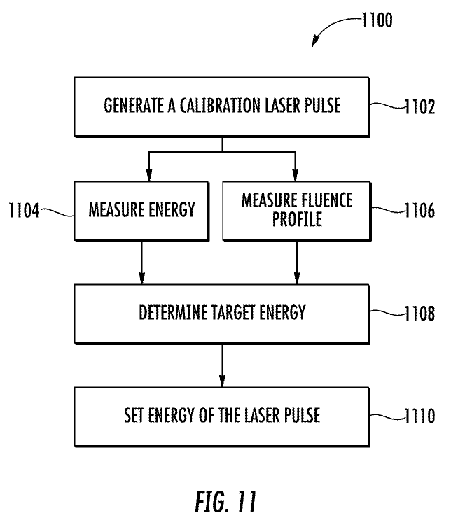

FIG. 11 illustrates an example method 1100 of controlling the energy of a laser pulse according to an embodiment. The method 1100 may be performed using the exemplary system 800 illustrated in FIG. 8. Referring to FIG. 11, the method 1100 includes generating a calibration laser pulse (e.g., using laser source 830), as indicated by step 1102. The method 1100 further includes measuring the energy of the calibration pulse (e.g., using energy meter 860), as indicated by step 1104, and measuring the fluence profile of the calibration laser pulse (e.g., using fluence profiler 840), as indicated by step 1106. For example, a beam divider (e.g., beam divider 805) may receive the calibration laser pulse, reflect a first portion of the calibration laser pulse toward a treatment plane and send a second portion of the calibration laser pulse toward an equivalent plane. The optical path from the laser source to the equivalent plane correlates to an optical path from the laser source to the treatment plane. The energy of the first portion of the calibration pulse and the fluence profile of the second portion of the calibration laser pulse are measured, as indicated by step 1106.

The energy of an ablation laser pulse produced by the laser source (e.g., laser source 830) is controlled (e.g., by processor 870) by either changing one or more system parameters (e.g., voltage) to cause the laser source to produce a laser pulse having a different energy (e.g., different than the measured energy) of the calibration pulse or maintaining (i.e., not adjusting) the one or more system parameters such that the the energy pulse settings remains the same. For example, the parameters may be maintained or changed based on the measured energy of the calibration pulse and the measured fluence profile of the calibration laser pulse.

The energy of an ablation laser pulse may be controlled by changing or maintaining one or more system parameters based on a target energy. For example, as shown at step 1108 in FIG. 11, the method 1100 further includes determining (e.g., using processor 870) a target energy of the laser pulse. The processor 870 uses the measured energy and fluence profile of the calibration laser pulse to generate an energy control signal (e.g., control signal 880). The energy control signal 880 may be sent to the laser source (e.g., to control circuitry 832 of laser source 830) to cause the laser source 830 to set the energy of the laser pulse according to the energy control signal 880, as indicated by step 1110.

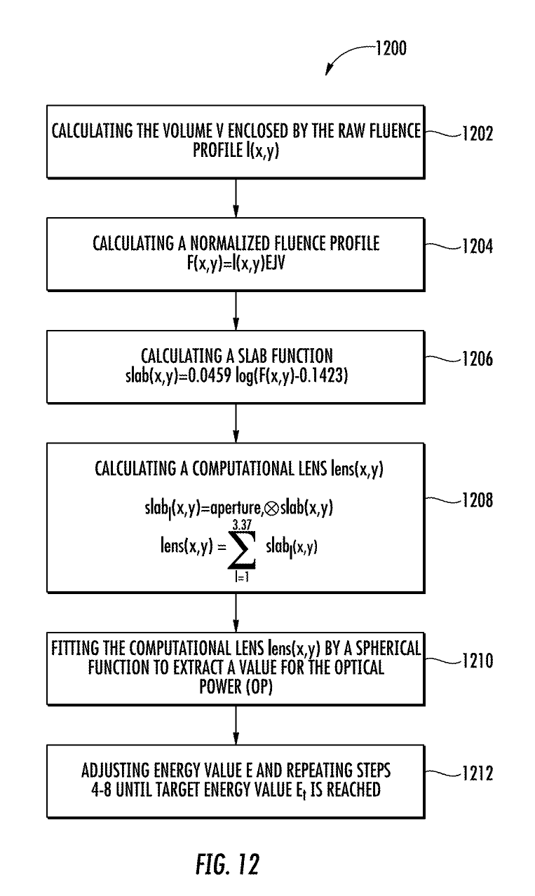

FIG. 12 illustrates an exemplary method 1200 of determining the target energy of the laser pulse at step 1108 of FIG. 11.

As indicated at step 1202 of FIG. 12, the method 1200 includes calculating the volume V enclosed by the raw fluence profile I(x,y) of the calibration laser pulse as,

V= [symbol] I(x,y)dxdy.

For instance, for the exemplary fluence profile shown in FIG. 10, the volume V is the volume under the envelope of the fluence surface I(x,y). The raw fluence profile I(x,y) may be measured in analog-to-digital-conversion (ADC) counts. The raw fluence profile may be recorded for an aperture size such as 6 mm in diameter or the like.

As indicated at step 1204, the method 1200 includes calculating a normalized fluence profile F(x,y) by dividing the raw fluence profile I(x,y) by the volume V and then multiplying the result by the measured energy E of the calibration laser pulse,

F(x,y)=I(x,y)E/V.

The energy E of the calibration laser pulse may be measured in mJ.

As indicated at step 1206, the method 1200 includes calculating a slab function using the following empirical equation,

slab(x,y)=0.0459 log(F(x,y)−0.1423).

The slab(x,y) may be measured in □m. The normalized fluence profile F(x,y) may be measured in mJ/cm2. In alternative embodiments, other empirical functions may be used for calculating the slab function. For example, an empirical function may be obtained from fitting calibration data in the fluence range of 150-180 mJ/cm2 by a linear regression method. For other fluence ranges, other formulas may be used.

As indicated at step 1208, the method 1200 includes calculating a computational lens lens(x,y) comprising a sequence of N computational slabs (e.g., 337 slabs). In some embodiments, the computational slabs may be constructed similar to the slabs in an ablated plastic lens, as described above in conjunction with FIG. 4. For example, each of the slabs may have the same thickness while their diameters vary sequentially from large to small. Each slab (slabᵢ(x,y)) is a convolution of a corresponding aperture function apertureᵢ(x,y) and the slab function slab(x,y),

slabᵢ(x,y)=apertureᵢ [symbol] slab(x,y).

The computation lens may be obtained by adding the N computational slabs,

$$lens ( x , y ) = {\sum }_{i = 1}^{n} {slab}_{i} ( x , y ) .$$

As indicated at step 1210, the method 1200 includes fitting the computational lens lens(x,y) by a spherical function to extract a value for the optical power (OP).

As indicated at step 1212, the method 1200 includes adjusting the energy value E may be adjusted and steps 1204-1210 may be repeated until a target optical power is achieved. For example, if the target optical power is −4 diopters, when the extracted value for the optical power in step 1210 is equal to −4 diopters or within a predetermined tolerance range of the target optical power, the energy value E is determined as the target energy value Eₜ. According to an embodiment, the processor 870 sends an energy control signal 880 to a control circuitry 832 of the laser source 830 to cause the parameters of the laser source 830 to set the energy (e.g., either maintain parameters or change parameter) of the laser pulse to the target energy value Eₜ.

In certain cases, the method 1200 of determining a target energy may not converge to a solution, or the target optical power cannot be achieved within the predetermined tolerance range. In those cases, the system performance limit may have been reached and may need further service. According to some embodiments, the system performance may be characterized in steps 1202 and 1204. It may be determined whether certain predetermined limits of fluence non-uniformity are met.

It is understood that the determining a target energy may include some or all of the above steps illustrated in FIG. 12. Determining a target energy may also include additional steps not illustrated in the method 1200 shown in FIG. 12.

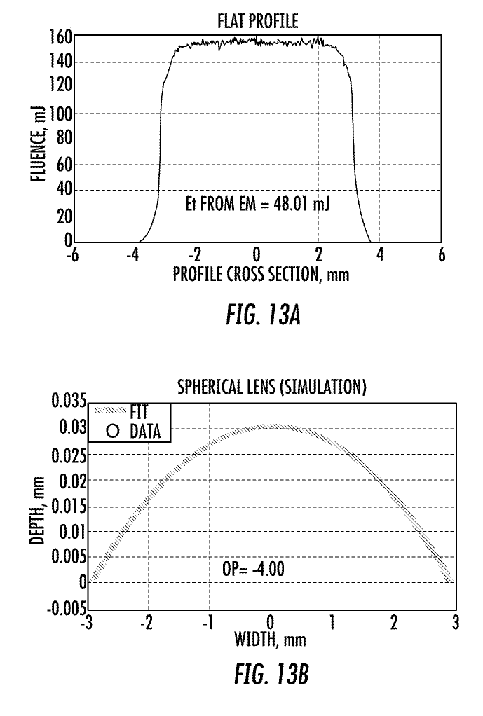

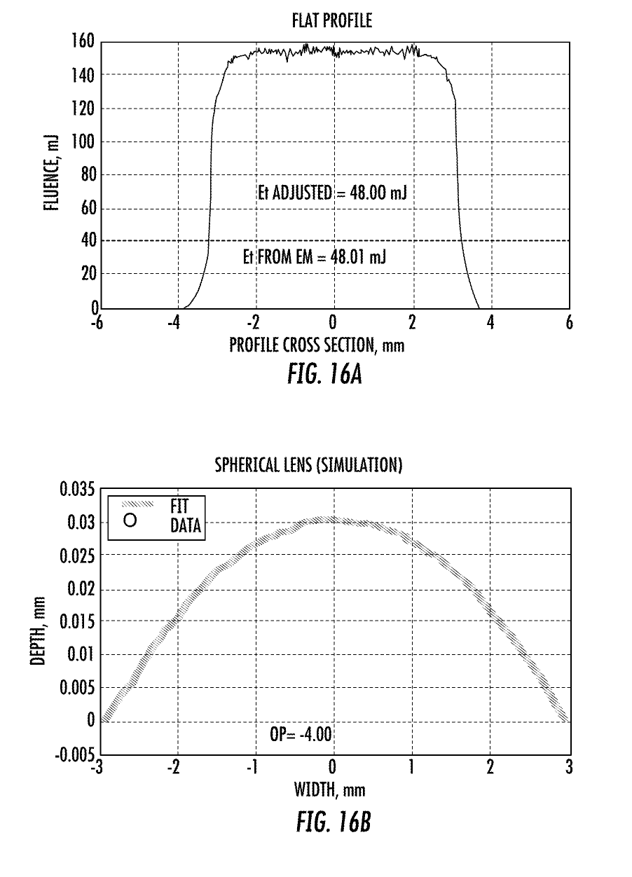

The following examples, shown in FIGS. 13A through 18B, are based on the fluence profiles shown in FIG. 4. The measured energy value from the energy meter is E=48.1 mJ. FIGS. 13-15 correspond to results before a target energy is determined and FIGS. 16-18 correspond to results after a target energy is determined.

FIG. 13A illustrates a cross section of an example fluence profile having a substantially flat shape within the spot area. FIG. 13B illustrates a computational spherical lens constructed according to the fluence profile shown in FIG. 13A. The energy of the calibration laser pulse is E=48.1 mJ. The computation spherical lens has an optical power of −4.00 diopters, substantially equal to the target optical power.

FIG. 14A illustrates a cross section of an example fluence profile having a concave shape within the spot area. FIG. 14B illustrates a computational spherical lens constructed according to the fluence profile shown in FIG. 14A. The energy of the calibration laser pulse is E=48.1 mJ. The computation spherical lens has an optical power of −3.93 diopters, which is lower than the target optical power of −4.0 diopters.

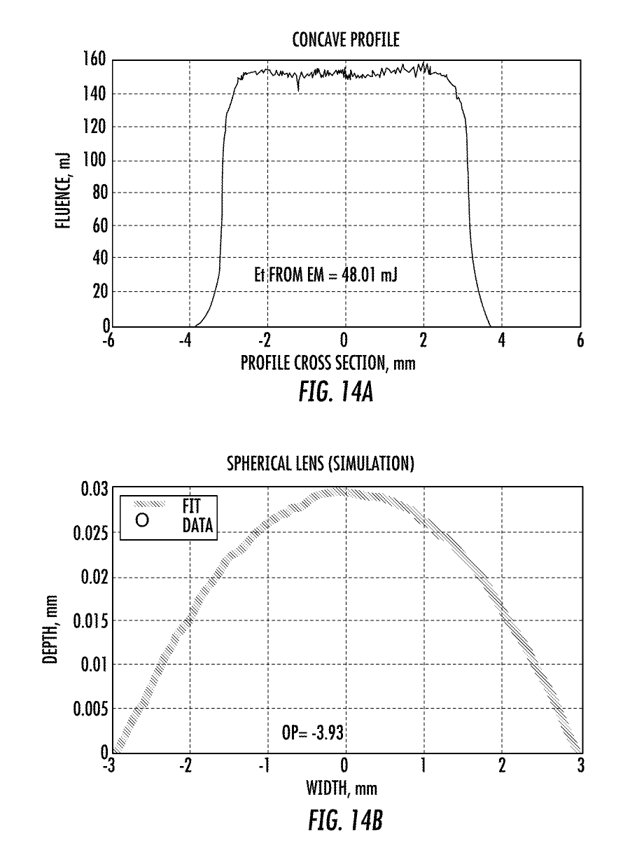

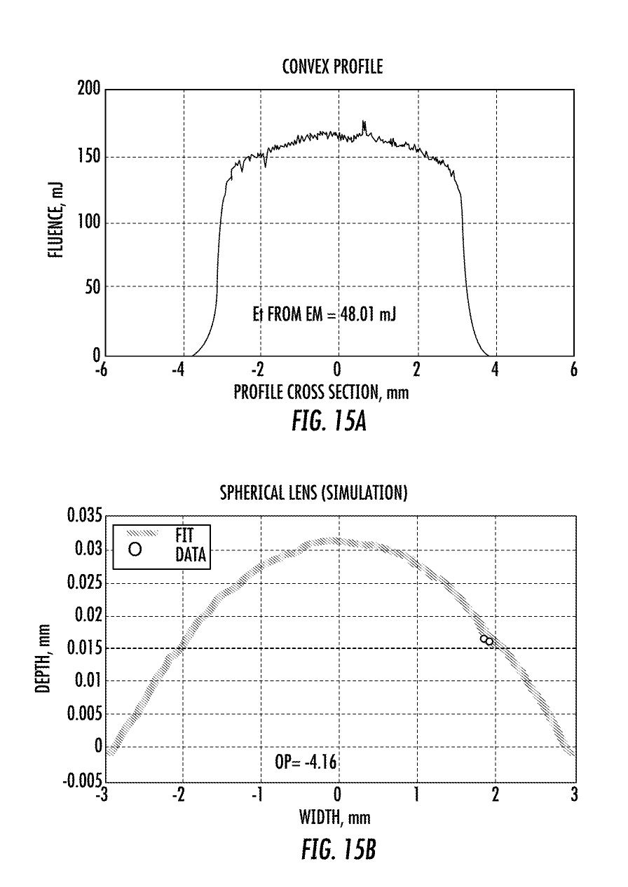

FIG. 15A illustrates a cross section of an example fluence profile having a convex shape within the spot area. FIG. 15B graphically illustrates a computational spherical lens constructed according to the fluence profile shown in FIG. 15A. The energy of the calibration laser pulse is E=48.1 mJ. The computation spherical lens has an optical power of −4.16 diopters, which is higher than the target optical power of −4.0 diopters

FIG. 16A graphically illustrates a cross section of an example fluence profile having a substantially flat shape within the spot area. FIG. 16B graphically illustrates a computational spherical lens obtained after determining a target energy according to an embodiments described herein. The computation spherical lens obtained has an optical power of −4.00 diopters, substantially equal to the target optical power. The corresponding target energy is 48.00 mJ, which is only slightly different from the measured energy of 48.01 mJ.

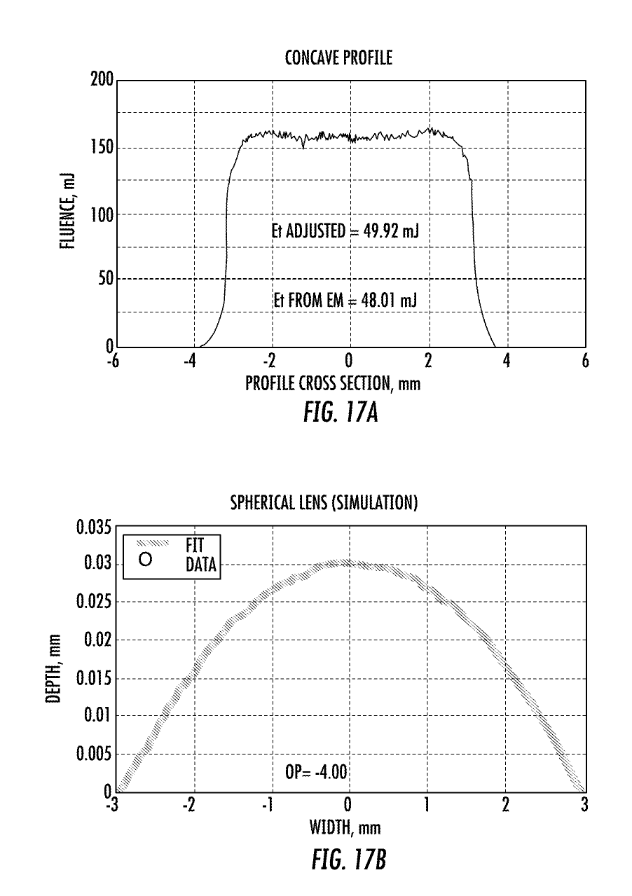

FIG. 17 shows a cross section of a fluence profile (top) of a calibration laser pulse that has a “concave” shape within the spot area, and a computational spherical lens (bottom) obtained after after a target energy is determined according to an embodiment. The computation spherical lens has an optical power of −4.00 diopters, substantially equal to the target optical power. The corresponding target energy Eₜ is 49.92 mJ. Thus, according to an embodiment, the processor may send an energy control signal to the control circuitry of the laser source to set the energy of the laser pulse at the target energy Eₜ=49.92 mJ.

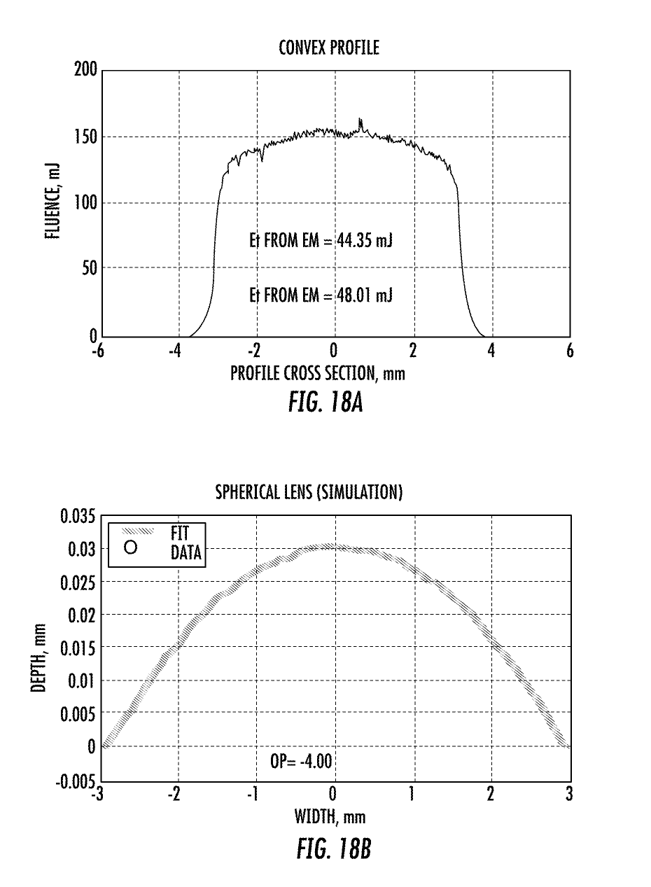

FIG. 18A graphically illustrates a cross section of an example fluence profile having a convex shape within the spot area. FIG. 18B graphically illustrates a computational spherical lens obtained after determining a target energy according to an embodiments described herein. The computation spherical lens obtained after after a target energy is determined has an optical power of −4.00 diopters, substantially equal to the target optical power. The corresponding target energy Eₜ is 44.35 mJ. Thus, according to an embodiment, the processor may send an energy control signal to the control circuitry of the laser source to set the energy of the laser pulse at the target energy Eₜ=44.35 mJ.

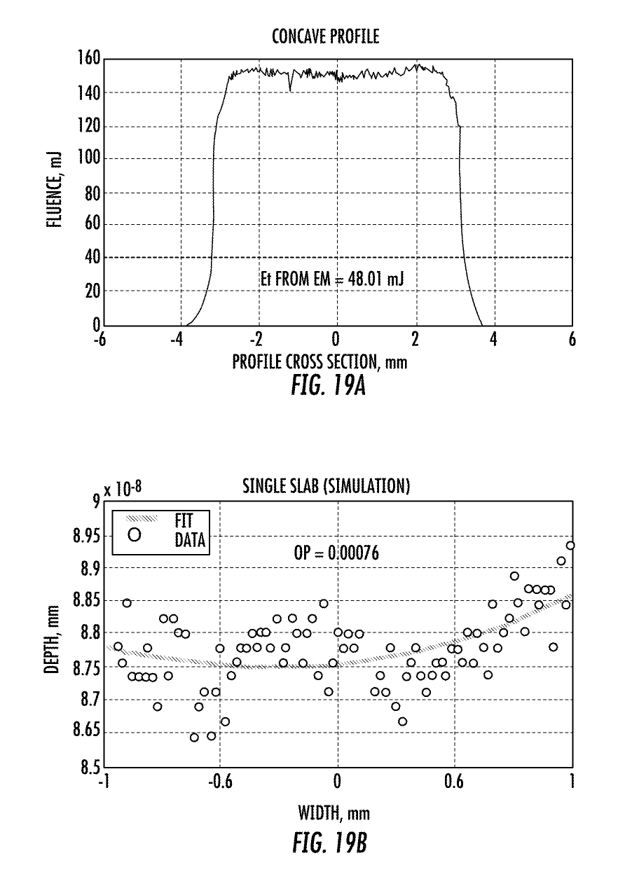

Other embodiments include calculating a single computational slab and fitting the computational slab by a spherical function to extract a value for the optical power of the single computational slab. FIG. 19A graphically illustrates a cross section of an example fluence profile having a concave shape within the spot area. FIG. 19B graphically illustrates a single computational slab obtained using the fluence profile shown in FIG. 19A. In this example, the extracted value for the optical power of the single computational slab is about 0.00076 diopters.

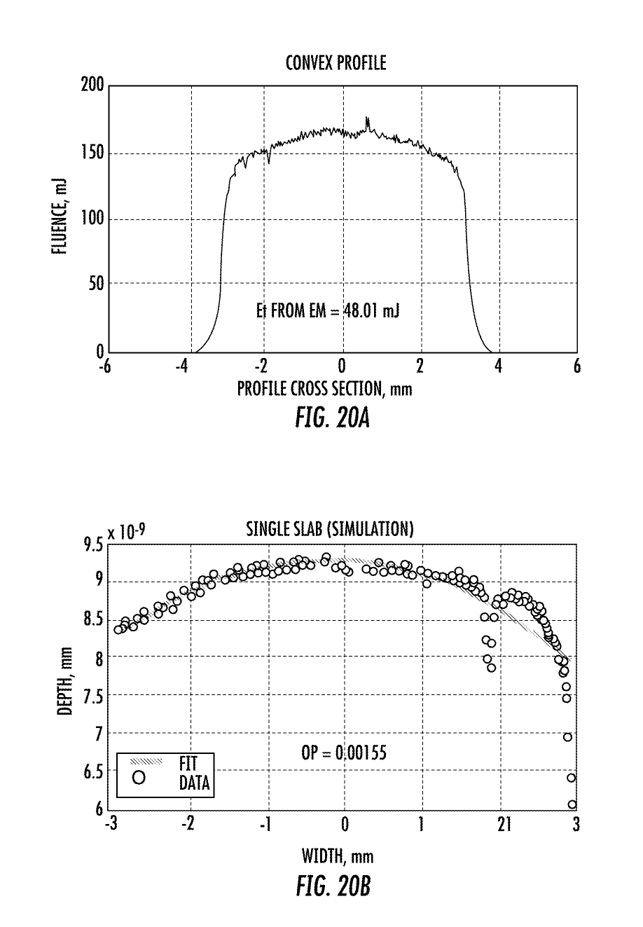

FIG. 20A graphically illustrates a cross section of a fluence profile having a convex shape within the spot area. FIG. 20B graphically illustrates a single computational slab obtained using the fluence profile shown in FIG. 20A. In this example, the extracted value for the optical power of the single computational slab is about −0.00155 diopters.

Embodiments described herein are not limited to the method described above in conjunction with FIG. 12. For example, in some embodiments, methods may include calculating a cylindrical computational lens or lenses of other shapes, and perform multivariate determination for multiple parameters that determine those shapes.

Embodiments described herein can be applied for other purposes. For example, embodiments can be used for alignment and maintenance in a laser vision correction system. Some embodiments can be used to evaluate the quality of a laser beam according to certain specified metric. The quality of the laser beam can, in turn, be used by service personnel as a feedback for preceding maintenance action(s). Accordingly, system alignment procedures may be simplified and the beam profile setting accuracy during the alignment procedure can be improved. Embodiments described herein can also be used for other energy profiles which may, or may not, be dependent on aperture size. In addition, embodiments may be utilized for real-time feedback based on configurations of the sensing devices (e.g., fluence profiler and energy meter) to improve the energy and fluence profile of a pulse. Embodiments can be utilized to account for ophthalmologic measurement and diagnostic system errors (i.e., basis functions for ablation) for laser vision treatment planning and for system performance quantification of an emitted energy profile. In some embodiments, performance may be defined as initial system characteristics and/or system degradation over time. Further, performance characterization can be utilized for feedback for adjustments in the laser correction treatment and or a quality metric when the system can no longer be used.

All patent filings (including patents, patent applications, and patent publications), scientific journals, books, treatises, technical references, and other publications and materials discussed in this application are incorporated herein by reference in their entirety for all purposes.

A variety of modifications are possible within the scope of embodiments described herein. A variety of parameters, variables, factors, and the like can be incorporated into the exemplary method steps or system modules. While the specific embodiments have been described in some detail, by way of example and for clarity of understanding, a variety of adaptations, changes, and modifications will be obvious to those of skill in the art. Although embodiments are described herein with specific reference to a wavefront system using lenslets, other suitable wavefront systems that measure angles of light passing through the eye may be employed. For example, systems using the principles of ray tracing aberrometry, tscherning aberrometry, and dynamic skiascopy may be used with embodiments described herein. The above systems are available from TRACEY Technologies of Bellaire, Tex., Wavelight of Erlangen, Germany, and Nidek, Inc. of Fremont, Calif., respectively. Embodiments embodiments described herein may also be practiced with a spatially resolved refractometer as described in U.S. Pat. Nos. 6,099,125; 6,000,800; and 5,258,791, the full disclosures of which are incorporated herein by reference. Treatments that may benefit from the embodiments described herein include intraocular lenses, contact lenses, spectacles and other surgical methods in addition to refractive laser corneal surgery.

Each of the calculations or operations discussed herein may be performed using a computer or other processor having hardware, software, and/or firmware. The various method steps may be performed by modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems will often employ a tangible media embodying machine-readable code with instructions for performing the method steps described above. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like. While the exemplary embodiments have been described in some detail, by way of example and for clarity of understanding, those of skill in the art will recognize that a variety of modification, adaptations, and changes may be employed.

The methods and apparatuses of the embodiments described herein may be provided in one or more kits for such use. The kits may comprise a system for determining a treatment for an eye of a patient, and instructions for use. Optionally, such kits may further include any of the other system components described in relation to embodiments described herein and any other materials or items relevant to embodiments described herein. The instructions for use can set forth any of the methods as described herein.

While the above provides a full and complete disclosure of exemplary embodiments described herein, various modifications, alternate constructions and equivalents may be employed as desired. Consequently, although the embodiments have been described in some detail, by way of example and for clarity of understanding, a variety of modifications, changes, and adaptations will be obvious to those of skill in the art. Accordingly, the above description and illustrations should not be construed as limiting the embodiments, which can be defined by the claims.

What is claimed is:

1. A laser pulse energy control system comprising:
a laser source;
a beam divider positioned to receive a calibration laser pulse produced by the laser source, the beam divider configured to reflect a first portion of the calibration laser pulse along a first optical path toward a first plane, and transmit a second portion of the calibration laser pulse along a second optical path toward a second plane;
an energy meter configured to determine an energy of the first portion of the calibration laser pulse at the first plane;
a fluence profiler configured to determine a fluence profile of the second portion of the calibration laser pulse at the second plane; and
a processor configured to control an energy of an ablation laser pulse produced by the laser source based on the fluence profile of the second portion of the calibration laser pulse and the energy of the first portion of the calibration laser pulse.

2. The system of claim 1, wherein the processor is further configured to control the energy of the ablation laser pulse by:
determining a target energy based on the fluence profile of the second portion of the calibration laser pulse and the energy of the first portion of the calibration laser pulse; and
causing the laser source to produce the ablation laser pulse having the target energy.

3. The system of claim 2, wherein the processor is further configured to determine the target energy by:
calculating an initial computational lens based on the fluence profile of the second portion of the calibration laser pulse and the energy of the first portion of the calibration laser pulse;
deriving an initial optical power of the initial computational lens by fitting the initial computational lens to a lens function;
obtaining an adjusted energy value based on the energy of the first portion of the calibration laser pulse, the initial optical power, and a predetermined target optical power;
calculating a target computational lens based on the fluence profile of the second portion of the calibration laser pulse and the adjusted energy value, wherein the target computational lens has an optical power that is substantially equal to the predetermined target optical power; and
determining the target energy based on the target computational lens.

4. The system of claim 3, wherein the processor is further configured to calculate the initial computational lens by:
calculating a volume enclosed by the fluence profile of the second portion of the calibration laser pulse;
calculating a normalized fluence profile by multiplying the fluence profile of the second portion of the calibration laser pulse by a ratio of the energy of the first portion of the calibration laser pulse and the volume;
calculating a slab function based on the normalized fluence profile;
calculating a sequence of computational slabs, wherein each respective computational slab is a convolution of the slab function with a corresponding aperture function; and
adding the sequence of computational slabs to obtain the initial computational lens.

5. The system of claim 4, wherein the corresponding aperture function for each of the sequence of computational slabs corresponds to a respective aperture diameter that sequentially varies from a large diameter to a small diameter for the sequence of computational slabs.

6. The system of claim 4, wherein each of the sequence of computational slabs has a same thickness with respect to each other.

7. The system of claim 4, wherein the slab function comprises a logarithm function.

8. The system of claim 4, wherein the slab function comprises an empirical function derived from a regression algorithm.

9. The system of claim 3, wherein the lens function comprises a spherical function.

10. The system of claim 3, wherein the lens function comprises a combination of a spherical function and a cylindrical function.

11. The system of claim 1, wherein the first plane is a treatment plane and the fluence profiler is located at the second plane, such that a length of the first optical path is equal to a length of the second optical path.

12. A laser pulse energy control method comprising:
receiving, at a processing device, an energy value corresponding to an energy of a first divided portion of a calibration laser pulse generated by a laser source, the energy being determined at a first plane;
receiving, at the processing device, fluence profile information corresponding to a fluence profile of a second divided portion of the calibration laser pulse generated by the laser source, the fluence profile being determined at a second plane; and
controlling, by the processing device, an energy of an ablation laser pulse produced by the laser source based on the fluence profile of the second divided portion of the calibration laser pulse and the energy of the first divided portion of the calibration laser pulse.

13. The method of claim 12, further comprising:
generating the calibration laser pulse using the laser source;
dividing the calibration laser pulse into a first divided portion and a second divided portion, such that the first divided portion travels along a first optical path toward the first plane and the second divided portion travels along a second optical path toward the second plane, wherein a length of the first optical path is equal to a length of the second optical path;
measuring the energy of the first divided portion of the calibration laser pulse at the first plane; and
measuring the fluence profile of the second divided portion of the calibration laser pulse at the second plane.

14. The method of claim 12, further comprising controlling the energy of the ablation laser pulse by:
determining a target energy based on the fluence profile of the second divided portion of the calibration laser pulse and the energy of the first divided portion of the calibration laser pulse; and
causing the laser source to produce the ablation laser pulse having the target energy.

15. The method of claim 14, further comprising determining the target energy by:
calculating an initial computational lens based on the fluence profile of the second divided portion of the calibration laser pulse and the energy of the first divided portion of the calibration laser pulse;
deriving an initial optical power of the initial computational lens by fitting the initial computational lens to a lens function;
obtaining an adjusted energy value based on the energy of the first divided portion of the calibration laser pulse, the initial optical power, and a predetermined target optical power;
calculating a target computational lens based on the fluence profile of the second divided portion of the calibration laser pulse and the adjusted energy value, wherein the target computational lens has an optical power that is substantially equal to the predetermined target optical power; and
determining the target energy based on the target computational lens.

16. The method of claim 15, further comprising calculating the initial computational lens by:
calculating a volume enclosed by the fluence profile of the second divided portion of the calibration laser pulse;
calculating a normalized fluence profile by multiplying the fluence profile of the second divided portion of the calibration laser pulse by a ratio of the energy of the first divided portion of the calibration laser pulse and the volume;
calculating a slab function based on the normalized fluence profile;
calculating a sequence of computational slabs, wherein each respective computational slab being a convolution of the slab function with a corresponding aperture function; and
adding the sequence of computational slabs to obtain the initial computational lens.

17. The method of claim 16, wherein the corresponding aperture function for each of the sequence of computational slabs corresponds to a respective aperture diameter that sequentially varies from a large diameter to a small diameter for the sequence of computational slabs.

18. The method of claim 17, wherein each of the sequence of computational slabs has a same thickness with respect to each other.

19. The method of claim 18, wherein the slab function comprises a logarithm function.

20. A computer processing device comprising:
memory configured to store energy values and fluence profile information;
a processor configured to:
receive an energy value corresponding to an energy of a first divided portion of a calibration laser pulse generated by a laser source, the energy being determined at a first plane;
receive fluence profile information corresponding to a fluence profile of a second divided portion of the calibration laser pulse generated by the laser source, the fluence profile being determined at a second plane; and
control an energy of an ablation laser pulse produced by the laser source by determining a target energy based on the fluence profile of the second portion of the calibration laser pulse and the energy of the first portion of the calibration laser pulse; and
generate a control signal for causing the laser source to produce the ablation laser pulse having the target energy,
wherein, in response to receiving the control signal, the energy of the ablation laser pulse is controlled by one of: (i) adjusting parameter settings such that the energy of the ablation laser pulse is adjusted to the target energy; and (ii) maintaining the parameter settings such that the energy of the ablation laser pulse is maintained at the target energy.
